(19) United States
(12) Patent Application Publication
(10) Pub. No.: US 2001/0041676 A1
(43) Pub. Date: Nov. 15, 2001

(54) Glycosides and orthoester glycosides of glucocorticoids and uses thereof

(76) Inventors: Michael F. Holick, Sudbury, MA (US); Halasya Ramanathan, Worcester, MA (US)

Correspondence Address: STERNE, KESSLER, GOLDSTEIN & FOX PLLC, 1100 NEW YORK AVENUE, N.W., SUITE 600, WASHINGTON, DC, 20005-3934, US

(21) Appl. No.: 09/759,371

(22) Filed: Jan. 16, 2001

Related U.S. Application Data
(60) Provisional application No. 60/176,007, filed on Jan. 14, 2000.

(51) Int. Cl. A61K 31/704; C07J 5/00

(52) U.S. Cl. 514/26; 536/5

(57) ABSTRACT

Serum levels of therapeutically useful glucocorticosteroids are substantially increased by oral administration of the corresponding glycoside or orthoester glycoside.

### CROSS REFERENCE TO RELATED APPLICATION

This claims priority to U.S. Provisional Application No. 60/176,007, filed Jan. 14, 2000, the contents of which are fully incorporated by reference herein.

### BACKGROUND OF THE INVENTION

1. Field of the Invention

The present invention relates to glycosides and orthoester glycosides of glucocorticosteroids and their use in therapy, especially in the treatment of such conditions as adrenal insufficiency, inflammation and the modulation of immune responses.

2. Related Art

Glucocorticosteroids are adrenal cortical hormones biosynthesized from cholesterol in the adrenal cortex. Glucocorticosteroids are typically characterized by having 21 carbon atoms, an α,β-unsaturated ketone in ring A, and an α-ketol group attached to ring D. They differ in the extent of oxygenation or hydroxylation at C-11, C-17 and C-19 (Rawn, “Biosynthesis and Transport of Membrane Lipids and Formation of Cholesterol Derivatives,” in Biochemistry, Daisy et al. (eds.), 1989, pg. 567).

An example of an important naturally occurring glucocorticoid is hydrocortisone. Hydrocortisone is synthesized from progesterone by successive hydroxylation reactions at the C-11, C-17 and C-21 by an enzyme complex containing a cytochrome P₄₅₀ component and adrenodoxin (Rawn, supra). Derivatives of hydrocortisone include hydrocortisone acetate, hydrocortisone cypionate, hydrocortisone sodium phosphate and hydrocortisone sodium succinnate.

There are a number of synthetic glucocorticoids which have further functionalisation at various places on the glucocorticosteroid skeleton and sometimes lack saturation in ring A. Some of the most common synthetic glucocorticosteroids include beclomethasone dipropionate, betamethasone, betamethasone acetate, betamethasone benzoate, betamethasone disodium phosphate, cortisone acetate, dexamethasone, dexamethasone sodium phosphate, fludrocortisone acetate, flunisolide, fluoncinolone acetonide, fluocinonide, flurandrenolide, deflazacort, methylprednisolone, methylprednisolone acetate, methylprednisolone sodium succinnate, prednisolone, prednisolone acetate, prednisolone sodium phosphate, prednisolone tebutate, prednisone, triamcinolone, triamcinolone acetonide, triamcinolone diacetate and triamcinolone hexaacetonide.

Glucocorticosteroids are of clinical interest, owing to the fact that they are useful for replacement therapy in adrenal insufficiency (e.g., Addison's disease and congenital hypoplasia). In addition, glucocorticosteroids are known to reduce inflammation in a variety of tissues and promote immunosuppression.

Glucocorticosteroids have, therefore, found therapeutic uses in the treatment of a number of diseases including inflammatory diseases of the lower bowel (e.g., Morbus Crohn and ulcerative colitis), non-infectious acute ocular inflammation (e.g., allergic blepharitis, iritis, uveitis, choroiditis, conjunctivitis and sympathetic ophthalmia), cerebral edema, infantile massive spasms, acute allergic disorders (e.g., rhinitis, acute bronchial asthma, status asthmaticus, obstructive pulmonary disease), arthritis, rheumatism, nephrotic syndrome, skin diseases (e.g., pruritus, atopic dermatitis, psoriasis, dermatitis herpetiformis, pemphigus, erythema multiforme, exfoliative dermatitis, mycosis fungoides and eczema), respiratory distress syndrome in infants and immune system diseases (e.g., systemic lupus erythromatosus, polyarteritis nodosa, temporal arteritis, Wegner's granulomatosis, polymyositis and polymyalgia rheumatica)—c.f. Nichols, “Hormones,” in Remington: The Science and Practice of Pharmacy-Volume II, Gennaro (ed.), 1995 (pp. 1064-1076).

U.S. Pat. No. 5,908,833 discloses a glucocorticosteroid (GCS) chemically bound to a sugar, having the general formula:

GCS¹—O—Sugar¹

for colon or ileum specific delivery of the glucocorticosteroid to inflamed bowel mucosa, as well as processes for their preparation, pharmaceutical preparations containing the compounds and the use of the compounds in therapy. This patent teaches that the ideal profile for local treatment of small bowel inflammation in Morbus Crohn (especially in resected patients with poor function of the ileo-cecal valve) is a glucocorticosteroid-glycoside releasing a potent glucocorticosteroid with very high first-pass metabolism in the liver. The glycoside is not active until it is cleaved in the small bowel, and it is anticipated that much higher local concentrations of active glucocorticosteroid can be reached at the bacterial front by localized cleavage in the small bowel. Thus, the side effects of the glucocorticosteroid can be avoided by direct delivery to the target area, where the glycoside is cleaved. High first pass metabolism then helps to reduce the side effects when diffusing away from the affected area. Preferred glucocorticosteroid-glycosides include the β-linked D-glucose 22R-epimer of budesonide.

Friend et al., (Drug Design and Delivery, (1990)) experiment with the idea that the gut flora have a role to play in breaking down glucosides of glucocorticosteroids, with the aim of targeting the colon.

Tozer et al. (Pharmaceutical Research, 1991) targets the colon with dexamethasone glucoside.

Friend and Chang (J. Med. Chem., 1985) provide the 21-yl β-D-glucosides and galactosides of dexamethasone, prednisolone, hydrocortisone and fluocortisone, and establish that there are differing rates and locations of hydrolysis of these compounds, with prednisolone glucoside being hydrolyzed to a substantial extent in the small intestine, compared to dexamethasone.

Although glucocorticosteroids have various useful attributes, in the treatment of inflammatory bowel disease and other conditions, they also have various undesirable side effects in the gut, and it is generally desirable that they be inactivated until they reach their target. In the art, glucocorticosteroid glycosides have been prepared with the idea of minimizing availability in general, only being cleaved at the target site. There is no free glucocorticosteroid to pass the gut wall, and the free compound is only generated by hydrolysis in the presence of the gut flora.

### SUMMARY OF THE INVENTION

Surprisingly, we have now found that serum levels of therapeutically useful glucocorticosteroids are actually substantially increased by oral administration of the corresponding glycoside or orthoester glycoside.

In a first aspect, the present invention provides a composition for the treatment of a condition treatable by the systemic administration of a glucocorticosteroid, characterized in that the said glucocorticosteroid is a derivative in the form of a glycoside or orthoester glycoside, or salt or ester of the derivative.

The present invention also relates to a compound of the Formula (I):

GCS—OR₃ (I)

wherein GCS is a glucocorticosteroid and R₃ is a straight or branched chain glycosidic residue containing 1-20 glycosidic units per residue, or R₃ is an orthoester glycoside moiety of the Formula (II):

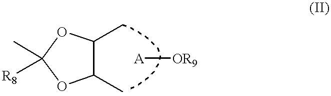

wherein A represents a glycofuranosyl or glycopyranosyl ring;

R₈ is hydrogen;

R₉ is hydrogen or a straight or branched chain glycosidic residue containing 1-20 glycosidic units per residue.

Preferably, the glucocorticosteroid is one that does not have a high first pass metabolism in the liver.

The present invention further provides compounds having Formula (III), which can be used to treat or prevent various conditions described herein:

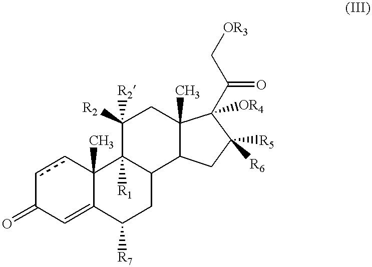

wherein the dotted line represents a single or a double bond;

R₁ is hydrogen or halogen;

R₂is OH;

R₂′ is hydrogen or alternatively R₂ and R₂′, together with the atom to which they are bound, are joined to form a carbonyl;

R₃ is a straight or branched chain glycosidic residue containing 1-20 glycosidic units per residue, or R₃ is an orthoester glycoside moiety of the Formula (II):

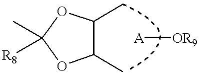

wherein A represents a glycofuranosyl or glycopyranosyl ring;

R₈ is hydrogen;

R₉ is hydrogen or a straight or branched chain glycosidic residue containing 1-20 glycosidic units per residue;

R₄ is hydrogen, —C(O)cycloalkyl-substituted alkyl, —C(O)aryl, —C(O)heterocyclo-substituted alkyl or —C(O)heteroaryl;

R₅ is hydrogen, alkyl, hydroxyl or alternatively R₄ and R₅, together with the atoms to which they are bound, are joined to form an acetonide;

R₆ is hydrogen or alkyl;

R₇ is hydrogen, halogen or alkyl.

The invention also relates to a method for the treatment or amelioration of adrenal insufficiency or an inflammatory or immune system disease which is treatable or ameliorated by systemic, transdermal, intranasal or inhalation administration of a glucocorticosteroid, comprising administering to an animal in need thereof, an effective amount of a compound having the Formulae (I) or (III).

The invention also relates to a method of preparing a compound of Formula (III) which comprises reacting a protected α-bromoglycoside or orthoester glycoside with a glucocorticosteroid in the presence of a base and cleaving the protecting groups.

### DETAILED DESCRIPTION OF THE PREFERRED EMBODIMENTS

Where the derivative is a glycoside, then it is preferred that it contain 1-20 glycosidic units.

Preferred glycosidic orthoester residues have the Formula (II):

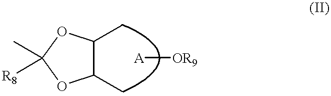

wherein A represents a glycofuranosyl or glycopyranosyl ring or amino derivative thereof;

R⁸ is hydrogen, C₁₋₄ alkyl, C₇₋₁₀ aralkyl, phenyl; or phenyl substituted by chloro, fluoro, bromo, iodo, C₁₋₄ alkyl or C₁₋₄ alkoxy; or naphthyl; and

R⁹ is hydrogen or a straight or branched chain glycosidic residue containing 1-20 glycosidic units per residue.

It is preferred that compounds of the present invention have less than 10 and, more preferably, 3 or less glycosidic units. Specific examples are those containing 1 or 2 glycosidic units in the glycoside residue, such as glucose and sucrose, with one being most preferred.

By glycosidic units are meant glycopyranosyl or glycofuranosyl, as well as their sulfates, amino sugar and/or deoxy derivatives. The configuration of each unit may be D or L, although D is generally preferred. The residues may be homopolymers, random or alternating polymers, or block copolymers of these monomers.

The glycosidic units have free hydroxy groups, or the hydroxy groups may be acylated, e.g. with a group R⁵—C═O)—, wherein R⁵ is hydrogen, C₁₋₆ alkyl, C₆₋₁₀ substituted or unsubstituted aryl or C₇₋₁₆ aralkyl. Preferably, the acyl groups are acetyl or propionyl. Other preferred R⁵ groups are phenyl, nitrophenyl, halophenyl, lower alkyl substituted phenyl, lower alkoxy substituted phenyl and the like or benzyl, lower alkoxy substituted benzyl and the like.

The glycopyranose or glycofuranose ring or amino derivative thereof may be fully or partially acylated or completely deacylated. The completely or partially acylated glycoside is useful as a defined intermediate for the synthesis of the deacylated material. Useful protecting groups include, but are not limited to, acetyl, benzoyl, nicotinoyl, benzyl, methyl and phenyl.

Among the possible glycopyranosyl structures are glucose, mannose, galactose, gulose, allose, altrose, idose, or talose. Among the furanosyl structures, the preferred ones are derived from fructose, ribose, arabinose or xylose. Among preferred diglycosides are sucrose, cellobiose, maltose, lactose, trehalose, gentiobiose, and melibiose. Among the triglycosides, the preferred ones may be raffinose or gentianose.

Preferred aminosugar derivatives are N-acetyl-D-galactosamine, N-acetyl-D-glucosamine, N-acetyl-D-mannosamine, N-acetylneuraminic acid, D-glucosamine, D-lyxosylamine, D-galactosamine, chondroitin, and the like. In addition, such active units as chondroitin sulfate and D-glucosamine sulfate may also be employed, as such sub-units independently have advantageous therapeutic osteopathic properties.

Where there are linked glycosidic units, i.e., there is a di or polyglycosidic residue, the individual glycosidic rings may be bonded by 1-1, 1-2, 1-3, 1-4, 1-5 or 1-6 bonds, most preferably 1-2, 1-4 and 1-6. The linkages between individual glycosidic rings may be α or β.

Alkyl groups may be straight, branched or cyclic and may conveniently be a C₁₋₁₀ alkyl, including octyl, nonyl, decyl, diethylhexyl, and, more preferably, C₁₋₆, such as methyl, ethyl, propyl, butyl, methylpropyl, t-butyl, pentyl, dimethylpropyl, hexyl, dimethylbutyl or ethylbutyl. Preferred alkyl groups contain 1 or 2 carbon atoms. Methyl and ethyl groups are particularly preferred, especially methyl.

In cycloalkyl-substituted alkyl groups, each group may be as specified for alkyl. Preferably the straight chain portion contains 1 or 2 carbon atoms, and is substituted by a cycloalkyl group containing between 3 and 7, preferably 5 or 6 carbon atoms. Most preferably, the cycloalkyl group is cyclopentyl.

Aryl groups generally have 6 to 14 carbon atoms having a single ring (e.g., phenyl) or multiple condensed rings (e.g., naphthyl or anthryl). Preferred aryl groups are phenyl and naphthyl, preferably phenyl.

Heteroaryl groups generally contain between 5 and 10 ring members, of which one, two or three may be selected from P, N, S and O. In general, preferred heteroatoms are N, and exemplary heteroaryls include pyrazolyl, imidazolyl and pyridinyl. Where such groups are substituted, then this may be as exemplified above for aryl.

Heterocyclic groups may generally be as exemplified for heteroaryl, provided that the ring is not completely unsaturated. Substituents may be as exemplified above, and suitable examples of heterocyclic groups include pyrrolidinyl and pyrimidinyl.

Halogen is generally F, Cl, Br or I, preferably Cl.

In preferred embodiments, there is a single bond between C-1 and C-2. In individually preferred species,

R₁, R₂′, R₄, R₅, R₆ and R₇ are hydrogen and R₂ is hydroxyl;

R₁, R₂′ and R₆ are hydrogen, R₂ is hydroxyl, R₄ and R₅, together with the atoms to which they are bound, form an acetonide and R₇ is a fluorine; or

R₁, R₂′, R₅, R₆ and R₇ are hydrogen, R₂ is hydroxyl and R₄ is —C(O)cycloalkyl substituted alkyl.

In other preferred embodiments, there is a double bond between C-1 and C-2. In individually preferred species,

R₁ is fluorine, R₂ is hydroxyl, R₂′, R₄, R₅ and R₇ are hydrogen and R₆ is methyl;

R₁ is fluorine, R₂ is hydroxyl, R₂′, R₅ and R₇ are hydrogen, R₆ is —C(O)aryl and R₆ is methyl;

R₁ is fluorine, R₂ is hydroxyl, R₂′, R₄, R₆ and R₇ are hydrogen and R₅ is methyl;

R₁, R₂′ and R₆ are hydrogen, R₂ is hydroxyl, R₄ and R₅, together with the atoms to which they are attached, form an acetonide and R₇is fluorine;

R₁ and R₇ are fluorine, R₂′ and R₆ are hydrogen, R₂ is hydroxyl and R₄ and R₅, together with the atoms to which they are attached, form an acetonide;

R₁, R₂′, R₄, R₅ and R₆ are hydrogen, R₂ is hydroxyl and R₇ is methyl;

R₁, R₂′, R₄, R₅, R₆ and R₇ are hydrogen and R₂ is hydroxyl;

R₁, R₄, R₅, R₆ and R₇ are hydrogen and R₂ and R₂′ form a carbonyl; or

R₁ is fluorine, R₂ is hydroxyl and R₂′, R₆ and R₇ are hydrogen and R₄ and R₅, together with the atoms to which they are attached, form an acetonide.

Individually, specifically preferred compounds include, but are not limited to, the 21-glycofuranosides, 21-glycopyranosides, straight chained or branched 21-oligoglycosides and orthoester glycosides of flurandrenolide, hydrocortisone, hydrocortisone cypionate, betamethasone, betamethasone benzoate, dexamethasone, flunisolide, fluoncinolone, methylprednisolone, prednisolone, prednisone and triamcinolone acetonide.

Especially preferred glucocorticosteroids include those which have low first-pass metabolism in the liver, including betamethasone, dexamethasone, triamcinolone acetonide, hydrocortisone, methylprednisolone, prednisolone and prednisone. One of ordinary skill in the art can determine whether a given glucocorticosteroid has low first-pass metabolism in the liver with no more than routine experimentation. By way of generalization, glucocorticosteroids which have low first-pass metabolism in the liver typically have a plasma half-life greater than about 1 hour.

Most preferred compounds according to the invention are the 21-glycofuranoside, 21-glycopyranoside, straight chained or branched 21-oligoglycosides and orthoester glycosides of prednisone and prednisolone.

Individually preferred glucocorticoids substituted in accordance with the invention are hydrocortisone, hydrocortisone acetate, hydrocortisone cypionate, hydrocortisone sodium phosphate, hydrocortisone sodium succinnate, beclomethasone dipropionate, betamethasone, betamethasone acetate, betamethasone benzoate, betamethasone disodium phosphate, cortisone acetate, dexamethasone, dexamethasone sodium phosphate, fludrocortisone acetate, flunisolide, fluoncinolone acetonide, fluocinonide, flurandrenolide, deflazacort, methylprednisolone, methylprednisolone acetate, methylprednisolone sodium succinnate, prednisolone, prednisolone acetate, prednisolone sodium phosphate, prednisolone tebutate, prednisone, triamcinolone, triamcinolone acetonide, triamcinolone diacetate and triamcinolone hexaacetonide.

The compounds useful in the practice of the invention contain at least one glycoside or orthoester glycoside residue connected to the 21-hydroxyl group of the glucocorticosteroid, e.g. prednisone or prednisolone.

The water soluble glycosidic derivatives of the aforementioned glucocorticosteroids may be obtained according to the general methods disclosed by Holick in U.S. Pat. No. 4,410,515,the contents of which are fully incorporated by reference herein.

The invention is related in particular to the synthesis of compounds of Formula (III). Examples of bases that can be used to form the desired linkage between the glucocorticosteroid and a bromoglycoside or orthoester glycoside include cadmium carbonate, silver carbonate, silver silicate, barium carbonate, lanthanum carbonate or oxalate, ytterbium carbonate or oxalate, and uranium carbonate or oxalate. In some cases, the glycoside or orthoester glycoside incorporates protecting groups. Examples of protecting groups include C₂₋₆ alkanoyl groups (e.g., the peracetate) and trialkylsilyl groups (e.g., t-butyldimethylsilyl and triisopropylsilyl). The reaction is carried out in an aprotic solvent, such as benzene, toluene, tetrahydrofuran, xylenes, chlorobenzene, dichlorobenzenes and the like.

The reaction temperature is from about 80 to 120° C. Preferably, the reaction temperature is about 110° C.

The reaction may be carried out for about 1 to 18 hours, preferably, about 4 hours or until TLC shows that the reaction is complete.

The thus formed glucocorticosteroid-glycoside or orthoester glycoside is then isolated and may be purified on a silica gel column. The protecting groups can then be removed and the glucocorticosteroid-glycoside or orthoester glycoside can be isolated and purified. When the protecting groups are C₂₋₆ alkanoyl, they may be removed by any known methods including treatment with alkali alkoxide in alcohol (e.g., sodium methoxide in methanol) or by treatment with a basic resin in alcohol (e.g., DOWEX™100-OH in methanol). In the case of trialkylsilyl and aryldialkylsilyl protecting groups, they may be removed in the presence of fluoride (e.g., tetrabutylammonium fluoride). In the case of benzyl groups, they may be removed by hydrogenation.

Representative examples of diseases treatable by compounds of the present invention are as listed hereinabove, and include, but are not limited to, replacement therapy in adrenal insufficiency (e.g., Addison's disease and congenital hyperplasia), non-infectious acute ocular inflammation (e.g., allergic blepharitis, iritis, uveitis, choroiditis, conjunctivitis and sympathetic ophthalmia), cerebral edema, infantile massive spasms, acute allergic disorders (e.g., rhinitis, acute bronchial asthma, status asthmaticus, obstructive pulmonary disease), arthritis, rheumatism, nephrotic syndrome, skin diseases (e.g., pruritus, psoriasis, dermatitis herpetiformis, pemphigus, erythema multiforme, exfoliative dermatitis, mycosis fungoides and eczema), respiratory distress syndrome in infants and immune system diseases (e.g., systemic lupus erythromatosus, polyarteritis nodosa, temporal arteritis, Wegner's granulomatosis, polymyositis and polymyalgia rheumatica).

The compounds of the present invention are preferably used where systemic action is indicated. These conditions are exemplified above, and do not include inflamed bowel mucosa.

Particularly preferred routes of administration of the compounds of the present invention are per os, such as elixirs, tablets and capsules, as exemplified below.

More generally, the compounds of the present invention can be administered in any appropriate pharmaceutically acceptable carrier for oral administration since the glucocorticosteroid-glycosides are biologically active upon oral administration. The compounds of the invention may also be administered in any appropriate pharmaceutical carrier for parenteral, intramuscular, transdermal, intranasal or inhalation administration. They can be administered by any means that treat or ameliorate adrenal insufficiency, non-infectious acute ocular inflammation, cerebral edema, infantile massive spasms, acute allergic disorders, arthritis, rheumatism, nephrotic syndrome, skin diseases, respiratory distress syndrome in infants and immune system diseases.

The dosage administered will depend on the age, health and weight of the recipient, kind of concurrent treatment, if any, frequency of treatment and the nature of the effect desired. An exemplary systemic daily dosage is about 0.1 mg to about 500 mg. Normally, from about 1.0 mg to 100 mg daily of the glycoside or orthoester glycoside, in one or more dosages per day, is effective to obtain the desired results. One of ordinary skill in the art can determine the optimal dosages and concentrations of active glucocorticosteroid compounds and orthoester glycoside compounds with only routine experimentation.

The compounds can be employed in dosage forms such as tablets and capsules for oral administration, as well as sterile liquid for formulations such as solutions or suspensions for parenteral use. A lipid vehicle can be used in parenteral administration. The compounds could also be administered via topical patches, ointments, gels or other transdermal applications. In such compositions, the active ingredient will ordinarily be present in an amount of at least 0.001% by weight based on the total weight of the composition, and not more than 50% by weight. An inert pharmaceutically acceptable carrier is preferable such as 95% ethanol, vegetable oils, propylene glycols, saline buffers, sesame oil, etc. Remington's Pharmaceutical Sciences, 18th Edition, Gennaro et al. (eds.), 1990, exemplifies methods of preparing pharmaceutical compositions.

Topical formulations for transdermal, intranasal or inhalation administration may be prepared according to methods well known in the art. For topical administration, the compounds may be applied in any of the conventional pharmaceutical forms. For example, the compounds may be administered as part of a cream, lotion, aerosol, ointment, powder, or drops. Ointments and creams may, for example, be formulated with an aqueous or oily base with the addition of suitable thickening and/or gelling agents. Such bases may include water and/or an oil such as liquid paraffin or a vegetable oil such as peanut oil or castor oil. Thickening agents which may be used include soft paraffin, aluminum stearate, cetostearyl alcohol, polyethylene glycols, wool-fat, hydrogenated lanolin, beeswax and the like.

Lotions may be formulated with an aqueous or oily base and will in general also include one or more of a stabilizing agent, thickening agent, dispersing agent, suspending agent, thickening agent, coloring agent, perfume and the like.

Powders may comprise any suitable powder base including talc, lactose, starch and the like. Drops may comprise an aqueous or non-aqueous base together with one or more dispersing agents, suspending agents, solubilizing agents and the like.

The compositions may further comprise one or more preservatives including bacteriostatic agents including methyl hydroxybenzoate, propyl hydroxybenzoate, chlorocresol, benzalkonium chloride and the like.

The topical compositions comprise from about 0.0001% to 5% by weight, preferably, 0.001 to 0.5% by weight, more preferably, 0.01 to 0.25% by weight.

The compounds may be administered as part of aerosolized pharmaceutical compositions to the oral airway passages and lungs by oral inhalation or intranasally by inhalation to treat, for example, upper and lower airway diseases and lower airway diseases such as inflammatory lung disease. The compounds may be administered using, for example, pressurized metered-dose inhalers which deliver aerosolized particles suspended in chlorofluorocarbon propellants such as CFC-11,CFC-12 or the non-chlorofluorocarbons, HFC-134A or HFC-227,with or without surfactants and suitable bridging agents; dry powder inhalers which are either breath activated or delivered by air or gas pressure such as the dry powder inhaler disclosed in PCT/US92/05225; the TURBOHALER™ (Astra Pharmaceutical Products, Inc.) or ROTAHALER™ (Allen & Hambury's) which may be used to deliver the compounds as a finely milled powder in large aggregates either alone or in combination with some pharmaceutically acceptable carrier (e.g lactose); and nebulizers. Reference is made to Remington's Pharmaceutical Sciences, 18th Edition, Gennaro et al. (eds.), 1990.

The compounds may also be administered in specific, measured amounts in the form of an aqueous solution or suspension by use of a pump spray bottle such as the bottles used to deliver VANCENASE AQ™ Nasal Spray. The aqueous solution or suspension may be prepared by admixing the compound with water and other pharmaceutically acceptable carriers. See PCT/US91/06249. The aqueous suspensions may contain from about 0.001 to 10.0 mg, preferably, 0.1 to 10.0 mg of the compound per gram of suspension. The aqueous solution or suspension may comprise auxiliaries and/or one or more excipients such as suspending agents (e.g. microcrystalline cellulose, sodium carboxymethyl-cellulose, hydroxypropylmethyl cellulose), hermectants (e.g. glycerine or propylene glycol), acids, bases or buffer substances for adjusting the pH (e.g. citric acid, sodium citrate, phosphoric acid, sodium phosphate as well as mixtures thereof); surfactants (e.g polysorbate 80) and antimicrobial preservatives (e.g. benzalkonium chloride, phenethyl alcohol and potassium sorbate).

For the treatment of allergic, non-allergic rhinitis and/or inflammatory diseases of the upper or lower airway passages to treat, for example, asthma or allergic or non-allergic rhinitis, the amount of compound which may be administered as an aqueous suspension and/or solution is in the range of 10 to 5000 mcg/day, 10 to 4000 mcg/day, 10 to 2000 mcg/day, 25 to 1000 mcg/day, 25 to 400 mcg/day, 25 to 200 mcg/day, 25 to 100 mcg/day, or 25 to 50 mcg/day, in single or divided doses. Other inflammatory diseases which may be treated include chronic obstructive pulmonary disease, granulomatous diseases of the lungs and lower airway passage, non-malignant proliferative disease of the lungs, e.g. idiopathic pulmonary fibrosis, hypersensitivity pneumonitis and bronchopulmonary dysplasia.

In the case of an inflammatory disease which is a skin disease, in one embodiment, the compound is administered topically to the skin of the animal. Preferably the skin disease is pruritus, psoriasis, dermatitis herpetiformis, pemphigus, erythema multiforme, exfoliative dermatitis, mycosis fingoides or eczema. Inflammatory diseases are preferably an inflammatory lung disease and the compound is administered by inhalation. More preferably, it is an upper airway disease and the compound is administered intranasally.

The compounds administered are substantially pure. The phrase “substantially pure” encompasses compounds created by chemical synthesis and/or compounds substantially free of chemicals which may accompany the compounds in the natural state, as evidenced by thin layer chromatography (TLC) or high performance liquid chromatography (HPLC).

Animals which may be treated according to the methods of the present invention include all animals which may benefit therefrom. Included in such animals are humans, although the invention is not intended to be so limited.

Having now generally described this invention, the same will be understood by reference to the following examples which are provided herein for purposes of illustration only and are not intended to be limiting unless otherwise specified.

### EXAMPLE 1

### Synthesis of 1,4-pregnadiene-17α-hydroxyl-21-O-1′-β′-glucopyranosyl-3,11,20-trione (prednisone-21-O-1′-β′-glucopyranoside) (5)

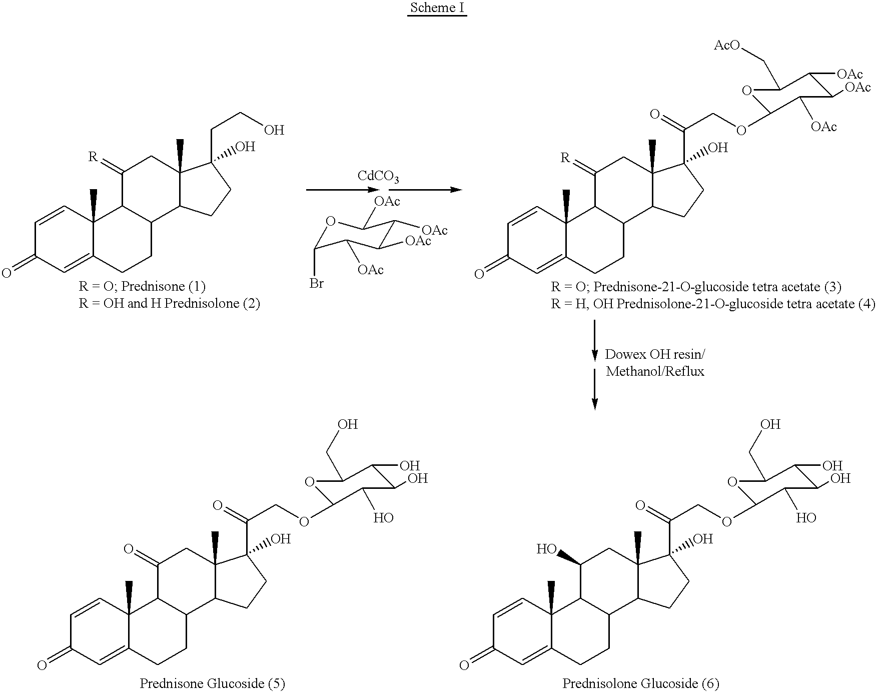

All of the starting materials were obtained from Sigma/Aldrich. Except for DOWEX-OH resin, all materials were used as obtained from the supplier. DOWEX-OH resin was washed with water and methanol prior to use.

The synthesis of prednisone and prednisolone glucosides was achieved selectively at the 21-hydroxy functionality, despite the presence of the 17α-tertiary hydroxyl and the 11-β-secondary hydroxyl groups of prednisone and prednisolone, respectively. The selectivity is attributable to the fact that the 17α-hydroxyl group of prednisone and the 11-β-hydroxyl group of prednisolone are more hindered relative to the 21-hydroxyl group of both compounds.

The reaction conditions used in this protocol were mild, not using any strong base or acid and, thus, avoiding the formation of any major side products. Acetobromo-α-D-glucopyranose, in refluxing toluene in the presence of insoluble cadmium carbonate, smoothly converted prednisone and prednisolone to their corresponding 21-O-1′-β′-glucosides (Scheme I).

A. Synthesis of 1,4-pregnadiene-17α-hydroxyl-21-O-1′-β-2′,3′,4′,6′-tetra-O-acetyl glucopyranosyl-3,11,20-trione (Prednisone-21-O-1′-β′-2′,3′,4′,6′-tetra-O-acetyl-glucopyranoside) (3)

A solution of prednisone (4.32 g, 12 mmol) and acetobromoglucose (9.6 g, 24 mmol) in toluene (150 ml) was stirred and heated to reflux under argon. Water was removed by azeotroping and cadmium carbonate (8.2 g, 48 mmol) was added to the reaction mixture. The argon atmosphere and refluxing were maintained for 4 hours, after which TLC showed there was no significant amount of starting material remaining. The mixture was chromatographed using silica gel and eluted with increasing polarity of hexane and ethyl acetate. At 80% ethyl acetate and 20% hexane, the title compound eluted (6.02 g, 71% yield). The remaining, more polar, starting prednisone eluted later (1.28 g). The glycosylated prednisone was crystallized from ethyl acetate-ether mixture to afford pure white solid.

¹H NMR (CDCl₃):

δ7.63 (d, C₁—H),

δ6.20 (d, C₂—H),

δ6.05 (br s, C₄—H),

δ5.25-2.25 (m, glucosyl and aliphatic protons, 14H),

δ2.00-2.25 (overlapping singlets of 4×CH₃—C=0 and aliphatic 4H),

δ1.75-1.50 (m, 4H),

δ1.48 (s, C₁₉—CH₃),

δ0.62 (s, C₁₈—CH₃).

Mass spectral analysis showed molecular ion at 711.2 amu (expected is 711.2).

B. Synthesis of 1,4-pregnadiene-17α-hydroxyl-21-O-1′-β′-glucopyranosyl-3,11,20-trione (Prednisone-21-O-1′-β′-glucopyranoside) (5)

A solution of prednisone-21-O-1′-β′-2′,3′,4′,6′-tetra-O-acetylglucopyranoside (5.9 g) in methanol (200 ml) was prepared. DOWEX 110-OH resin (6.5 g) was added and the mixture was refluxed under inert atmosphere for 5 hours. The resin was filtered off and the product was isolated after evaporating the methanol under reduced pressure. The glucoside was recrystallized from methanol-ethyl acetate. The yield of the title glucoside (5) was 4.3 g (almost quantitative yield).

¹H NMR (CDCl₃):

δ7.70 (d, C₁—H),

δ6.10 (d, C₂—H),

δ6.00 (br s, C₄—H),

δ4.90 (d, C₂₁—H),

δ4.40 (d, C₂₁—H),

δ4.20 (d, J_{1,2}=8 Hz; anomeric H),

δ3.90-1.55 and 1.30-1.00 (m, 19H, glucosyl H and aliphatic H),

δ1.45 (s, C₁₉—CH₃),

δ0.65 (s, C₁₈—CH₃).

Mass spectrum showed molecular ion with Na⁺ at 543.2 amu (expected is 543.6).

### EXAMPLE 2

### Synthesis of 1,4-pregnadiene-11β-17α-diol-21-O-1′-β′-glucopyranosyl-3,20-dione (prednisolone-21-O-1′-β′-glucopyranoside) (6)

A. Synthesis of 1,4-pregnadiene-11β-17α-diol-21-O-1′-β′-2′,3′,4′,6′-tetra-O-acetyl glucopyranosyl-3,20-dione (prednisolone-21-O-1′-β′-2′,3′,4′,6′-tetra-O-acetyl-glucopyranoside) (4)

To toluene (175 ml) was added prednisolone (5.5 g, 15.27 mmol) and acetobromoglucose (9.4 g, 1.5 equivalents). The mixture was heated to reflux and water was removed by azeotroping. Cadmium carbonate (7.89 g, 3 equivalents) was introduced under argon and the refluxing was continued for a period of 4 hours more. The reaction mixture was cooled and chromatographed on silica gel using ethyl acetate-hexane mixture. The title compound (4) eluted earlier than the starting prednisolone. Recrystallization from ether-ethyl acetate yielded 4.2 g of product (4).

¹H NMR (CDCl₃):

δ7.68 (d, C₁—H),

δ6.20 (d, C₂—H),

δ6.05 (br s, C₄—H),

δ5.25-2.25 (m, glucosyl and aliphatic H, 17H),

δ2.25-1.75 (overlapping singlets of 4×CH₃CO and 4H multiplet),

δ1.50-1.75 (m, 4H, aliphatic H),

δ1.48 (s, C₁₉—CH₃),

δ0.63 (s, C₁₈—CH₃).

Mass spectrum showed molecular ion at 13.3 amu (expected is 713.7).

B. Synthesis of 1,4-pregnadiene-11β-17α-diol-21-O-1′-β′-glucopyranosyl-3,20-dione (prednisolone-21-O-1′-β′-glucopyranoside) (6)

4.2 g of compound 4 was added to a suspension of DOWEX 110-OH resin (6.8 g) in methanol (100 ml) and heated to reflux, which was maintained for 4 hours. After filtering off the resin, the solvent was removed at reduced pressure to afford a crystalline product (6) (2.04 g, 68% yield).

¹H NMR (CD₃OD):

δ7.45 (d, C₁—H),

δ6.20 (d, C₂—H),

δ5.95 (br s, C₄—H),

δ4.90 (d, C₂₁—H),

δ4.45 (d, C₂₁—H),

δ4.30 (br s, 1H),

δ4.25 (d, J_{1,2}=8 Hz; anomeric H),

δ1.45 (s, C₁₉—CH₃),

δ3.80-0.90 (m, glucosyl H and aliphatic H, C₂₀—H),

δ0.80 (s, C₁₈—CH₃).

Mass spectrum showed the molecular ion at 545.2 amu for the Na⁺ salt (expected for the Na⁺ salt is 522.6).

### EXAMPLE 3

1. Alternative Synthesis of Prednisolone Glucoside

Prednisolone glucoside was synthesized from acetobromoglucose and prednisolone using silver silicate as the reagent for the conjugation.

A. Preparation of silver silicate:

A solution of silver fluoride (25 g) in water (140 ml) was prepared and filtered, to remove any metallic silver, and was then degassed with argon. A solution of sodium metasilicate (71 g) in water (420 ml) was filtered, to remove particles, and then degassed with argon. Silver fluoride solution was slowly added with vigorous stirring to the sodium metasilicate solution. After this addition, the mixture was stirred for a further period of 30 minutes, and then the solution was filtered off. The yellow precipitate was then washed once with 400 ml water and then once with methanol-acetone mixture (1:1, about 250 ml) and dried under vacuum at room temperature in the absence of light. Silver fluoride prepared by this method is almost quantitative and was used immediately in the reactions.

B. Synthesis of 21-β-O-glucopyranosyl-11(β),17-dihydroxypregna-1,4-diene-3,20-dione (prednisolone-21-O-β-glucoside)

To a stirred solution of prednisolone (5 g), chloroform (180 ml) and molecular sieves (20 g) under argon atmosphere was added silver silicate (7 g), prepared in A above. The mixture was stirred for a further period of 30 minutes at room temperature, and then acetobromoglucose (10 g) was introduced in chloroform (30 ml), in one lot. The mixture was stirred till there was no further reaction. The product was isolated by filtering off the solids and evaporating the solvents at reduced pressure, followed by a silica gel flash column chromatography using ethyl acetate and hexane mixtures. The crude conjugated tetraacetate was used in the subsequent deacetylation sequence with methanol (150 ml) and water (10 ml) using Dowex-550-OH resin (40 g). The mixture was brought to reflux and stirred for 1 hour and 30 minutes by which time all the starting material had been consumed, as determined by TLC analysis using ethyl acetate. The filtrate was concentrated under reduced pressure and the crude gum was lyophilized to obtain pure white solids (3.26 g).

2. pH stability testing of prednisolone glucoside:

The acid stability of prednisolone glucoside was qualitatively assessed by thin layer chromatography (TLC).

Prednisolone glucoside was suspended in pH 1.0 (freshly prepared from 10 M HCl solution) at a concentration of 20 mg/ml and stirred as a turbid solution at room temperature for 72 hours. At 24 and 48 hours, 100 μl aliquots were removed and diluted with about 200 μl of methanol to make a homogeneous solution. Approximately 50 μl was applied to a TLC plate (Sigma-Aldrich #Z 29298-2, made by Merck—silica TLC plates) with several repeated small applications. As controls, standard applications of 10 μg of fresh prednisolone glucoside, dissolved in 100 μl of methanol, and about 10 μg of prednisolone, in 50 μl of methanol, were applied at separate spots. Several repeated small applications were used to minimize the size of applied spot.

The TLC plates were initially eluted with ethyl acetate and then illuminated with the Aldrich's hand held model UV source (short UV light source used, Spectroline #1281657). The TLC plates were then dried, reeluted with methanol and then illuminated again with the UV source.

Under the initial chromatography conditions (ethyl acetate), prednisolone glucoside remained at the origin and less polar materials such as prednisolone eluted. At both the 24-, 48-hour and 72 hrs time points, there were no visible spots that were less polar than prednisolone glucoside, no visible material that moved with standard prednisolone and no apparent decrease in the intensity of the spots at the origin. Under the second chromatography conditions (i.e. methanol), the prednisolone glucoside was eluted. At the 24-, 48-, and 72-hour time points, material eluted the same as the standard prednisolone glucoside with no other spots being observed and no apparent loss of intensity of the eluted material

Accordingly, the above data show qualitatively that prednisolone glucoside is stable at pH 1.0 for up to 72 hours.

3. HPLC assay of prednisolone and prednisolone glucoside treated animals:

1 ml of rat serum was introduced into Varian-abselut (3 ml) cartridge (Part number SP12103100; analytical sample preparation and solid phase extraction use cartridge). Suction was applied and the cartridge was washed with 3 ml of water followed by 2 ml of water. The cartridges were dried under suction for 5 minutes and a jet stream of argon was blown to dry the trace amounts of water. The cartridge was washed once with 3 ml of hexane and the steroidal portion was eluted with 5 ml of methanol. The methanol portion was dried under nitrogen. The dried extract, which contains prednisolone or the glucoside, was constituted with 300 μl of ethyl acetate-methanol mixture (290 μl of ethyl acetate and 10 μl of methanol). 100 μl injections were made using manual injections in the HPLC instrument.

Hewlett Packard-Agilent 1100 series instrument with UV detector G1315A and a manual injector with a 100 μL loop were used in the assay. The samples were monitored at 254 and 250 nm.

The solvents used in the elution were a mixture of methanol (2 parts), ethyl acetate (42 parts) and hexane (56 parts). The mixture was run at a flow rate of 1 ml per minute. The column used for the separation was a normal phase silica column from Waters SPHERISORB^{R} 5 μM silica (4.6 mm×250 mm) analytical column with part #PSS 830115.

The serum prednisolone and prednisolone glucoside levels were assessed from the animals dosed with either 20 mg of prednisolone or 29 mg of prednisolone glucoside (20 mg equivalent). The animals were sacrificed after 30 minutes and 60 minutes.

4. Pharmacokinetics of prednisolone and prednisolone-glycoside

Comparison of rat serum levels of prednisolone after administering prednisolone (20 mg) and prednisolone glucoside (29 mg). In Table 1, below, serum levels of prednisolone and prednisolone glucoside were determined.

TABLE 1

| | | | Prednisolone- |
|---|---|---|---|
| | | | glucoside group |
| | Prednisolone group | Prednisolone-glucoside | (29 mg) |
| | (20 mg) | group (29 mg) | prednisolone- |
| time | Prednisolone ng/ml | Prednisolone ng/ml | glycoside ng/ml |
| 30 mins | 349.5 | 861 | 639 |
| 60 mins | 156.3 | 349.5 | 0 |

The serum prednisolone levels were calculated based upon the HPLC analyses of prednisolone and prednisolone glucoside. These values are calculated based upon the mAU and area values calculated against the standards.

From the foregoing description, one skilled in the art can easily ascertain the essential characteristics of this invention, and without departing from the spirit and scope thereof, can make various changes and modifications of the invention to adapt it to various usages and conditions without undue experimentation. All patents, patent applications and publications cited herein are incorporated by reference in their entirety.

What is claimed is:

1. A composition for the treatment of a condition treatable by the systemic administration of a glucocorticosteroid, comprising that glucocorticosteroid and a pharmaceutically acceptable carrier therefor, characterized in that the glucocorticosteroid is a derivative in the form of a glycoside or orthoester glycoside, or salt or ester of the derivative.

2. The composition of claim 1, wherein the glucocorticosteroid has the Formula (III): 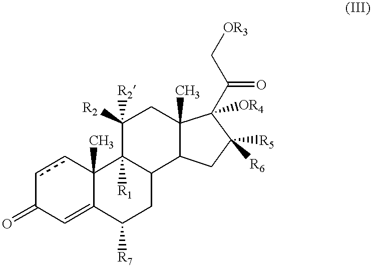
wherein the dotted line represents a single or a double bond;
R₁ is hydrogen or halogen;
R₂ is OH;
R₂′ is hydrogen or alternatively R₂ and R₂′, together with the atom to which they are bound, are joined to form a carbonyl;
R₃ is a straight or branched chain glycosidic residue containing 1-20 glycosidic units per residue, or R₃ is an orthoester glycoside moiety of the Formula (II): 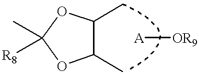
wherein A represents a glycofuranosyl or glycopyranosyl ring;
R₈ is hydrogen;
R₉ is hydrogen or a straight or branched chain glycosidic residue containing 1-20 glycosidic units per residue;
R₄ is hydrogen, —C(O)cycloalkyl-substituted alkyl, —C(O)aryl, —C(O)heterocyclo-substituted alkyl or —C(O)heteroaryl;
R₅ is hydrogen, alkyl, hydroxyl or alternatively R₄ and R₅, together with the atoms to which they are bound, are joined to form an acetonide;
R₆ is hydrogen or alkyl;
R₇ is hydrogen, halogen or alkyl.

3. The composition of claim 1, wherein said derivative is a glycoside containing 1-20 glycosidic units.

4. The composition of claim 1, wherein said derivative is a glycosidic orthoester having the Formula (II): 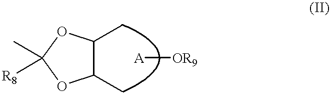
wherein A represents a glycofuranosyl or glycopyranosyl ring or amino derivative thereof;
R⁸ is hydrogen, C₁₋₄ alkyl, C₇₋₁₀ aralkyl, phenyl; or phenyl substituted by chloro, fluoro, bromo, iodo, C₁₋₄ alkyl or C₁₋₄ alkoxy; or naphthyl; and
R⁹ is hydrogen or a straight or branched chain glycosidic residue containing 1-20 glycosidic units per residue.

5. The composition of claim 1, wherein said derivative is a monoglycoside.

6. The composition of claim 5, wherein said glycosidic unit is a glucoside.

7. The composition of claim 1, wherein said glucocorticosteroid is one that does not have a high first pass metabolism in the liver.

8. The composition of claim 1, wherein said glucocorticosteroid is betamethasone, dexamethasone, triamcinolone acetonide, hydrocortisone, methylprednisolone, prednisolone or prednisone.

9. The composition of claim 1, wherein said glucocorticosteroid is prednisone-21-O-1′-β′-glucopyranoside.

10. The composition of claim 1, wherein said glucocorticosteroid is prednisolone-21-O-1′-β′-glucopyranoside.

11. A glucocorticosteroid derivative in the form of a glycoside or orthoester glycoside, or salt or ester of thereof, wherein said glucocorticosteriod does not have a high first pass metabolism in the liver.

12. The glucocorticosteroid derivative according to claim 11, having the formula: 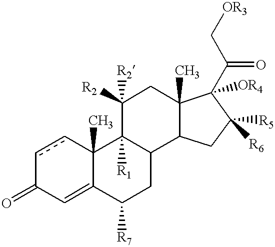
wherein the dotted line represents a single or a double bond;
R₁ is hydrogen or halogen;
R₂ is OH;
R₂′ is hydrogen or alternatively R₂ and R₂′, together with the atom to which they are bound, are joined to form a carbonyl;
R₃ is a straight or branched chain glycosidic residue containing 1-20 glycosidic units per residue, or R₃ is an orthoester glycoside moiety of the Formula (II): 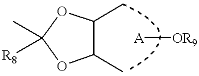
wherein A represents a glycofuranosyl or glycopyranosyl ring;
R₈ is hydrogen;
R₉ is hydrogen or a straight or branched chain glycosidic residue containing 1-20 glycosidic units per residue;
R₄ is hydrogen, —C(O)cycloalkyl substituted alkyl or —C(O)aryl;
R₅ is hydrogen, alkyl, hydroxyl or alternatively R₄ and R₅, together with the atoms to which they are bound, are joined to form an acetonide;
R₆ is hydrogen or alkyl; and
R₇ is hydrogen, halogen or alkyl.

13. The composition of claim 1, wherein said condition is adrenal insufficiency.

14. The composition of claim 13, wherein said condition is associated with Addison's disease or congenital hyperplasia.

15. The composition of claim 1, wherein said condition is an inflammatory or immune system disease.

16. The composition of claim 15, wherein said condition is a non-infectious acute ocular inflammation, cerebral edema, infantile massive spasms, an acute allergic disorder, arthritis, rheumatism, nephrotic syndrome, a skin disease, respiratory distress syndrome in infants and an immune system disease.

17. A method for the treatment or amelioration of an inflammatory disease, comprising administering by topical, intranasal or inhalation to an animal in need thereof, an effective amount of the compound of the glucocorticosteroid derivative of claim 11.

18. The method of claim 17, wherein said glucocorticosteroid derivative is administered as part of a pharmaceutical composition comprising a pharmaceutically acceptable carrier therefor.

19. A method for the treatment or amelioration of an inflammatory disease, comprising the oral administration to an animal in need thereof, an effective amount of the compound of the glucocorticosteroid derivative of claim 11.

20. The method of claim 19, wherein said glucocorticosteroid derivative is administered as part of a pharmaceutical composition comprising a pharmaceutically acceptable carrier therefor.
